(19) United States
(12) Patent Application Publication
(10) Pub. No.: US 2003/0175685 A1
(43) Pub. Date: Sep. 18, 2003

(54) Methods for identifying peptides which modulate a biological process

(75) Inventor: Malcolm L. Gefter, Lincoln, MA (US)

Correspondence Address: LAHIVE & COCKFIELD, 28 STATE STREET, BOSTON, MA, 02109, US

(73) Assignee: Praecis Pharmaceuticals Inc., Waltham, MA (US)

(21) Appl. No.: 10/080,532

(22) Filed: Feb. 22, 2002

Related U.S. Application Data
(60) Provisional application No. 60/270,968, filed on Feb. 22, 2001.

(51) Int. Cl. C12Q 1/70; G01N 33/53; G01N 33/567; G01N 33/554; G01N 33/569

(52) U.S. Cl. 435/5; 435/7.1; 435/7.32; 435/7.21; 530/324

(57) ABSTRACT

The invention provides methods and compositions for screening and identifying peptides which modulate a biological process in an organism, cell or tissue. The present invention further provides methods of using the identified peptides or analogues thereof to treat a disease or condition associated with an aberrant biological process in a subject.

### RELATED APPLICATIONS

This application claims priority to U.S. Provisional Patent Application Serial No. 60/270,968 filed Feb. 22, 2001, the entire contents of which are incorporated herein by reference.

### BACKGROUND OF THE INVENTION

Recent advances in methods for producing peptide libraries have provided vast numbers of peptides for screening for biological activity. Such methods include both biological methods and chemical synthetic methods. For example, peptides can be expressed by bacteriophage and presented at the phage surface for biological screening. Such libraries can include on the order of 10⁶ to 10¹² distinct members, and can include sequences which are random or biased, for example, with certain fixed residues or certain positions occupied only by one of a subset of possible residues. Such libraries provide a powerful method for identifying biologically active compounds.

One process for identifying compounds within a peptide or small molecule library having potential pharmaceutical activity involves screening compound libraries to identify library members which bind a target biomolecule, usually a protein. Generally, the target biomolecule is known or believed to be involved in a disease process. Compounds which bind the target biomolecule can then be evaluated in a functional screen, in which the effect of the compound on the function of the target is assessed. Such a screen can be a cell-free assay, in which the ability of the compound to modulate a molecular event, such as enzyme activity or ligand binding, is measured, or a cell based screen in which the ability of the compound to modulate a cellular activity is measured. The rate-determining factor in this method is the identification of target biomolecules which play a role in a particular disease process. The vast array of information available from efforts to sequence the human genome must be coupled to information does not decrease the need to validate the encoded proteins as therapeutic targets. The need to know at least some of the molecular details of a biological process associated with a disease state is a significant bottleneck in the development of new drugs.

One approach to the investigation of complex biological systems is to use combinatorial chemistry to synthesize diverse compound libraries that are screened for phenotypic effects in cells. Just as screens for the phenotypic effects of mutations served as an initial step in the characterization of basic metabolic and regulatory pathways in lower organisms several decades ago (i.e. in fungi and bacteria), it is believed that this approach may provide powerful means of examining the highly complex regulatory networks and pathways in mammalian cells. There are two crucial components to such an approach: (i) establishment of screening assays that allow phenotypic analysis of several million compounds, and (ii) development of highly diverse compound libraries in a format that allows molecular identification of the effective compound (deconvolution).

Both of these requirements are inadequately met by current technologies. The largest deconvolutable combinatorial chemical libraries that presently exist in tenable screening formats constitute one to two million compounds (Tan et al.(1998) PNAS 95(8):4247-52). Moreover, although phage display libraries represent a greater source of combinatorial diversity (i.e. 10⁹ different molecules in libraries composed of seven random natural amino acids), screening of these libraries is limited to evaluation of binding to known and specified target molecules. Screening only for binding does not immediately consider whether ligand binding affects a function of the target. In addition, since foreknowledge of a particular pathway and its components is required for the design of such binding screens, this approach is applicable only to targets within relatively well understood pathways.

Accordingly, the need still exists for improved methods which facilitate the identification of compounds capable of modulating biological processes associated with a disease state.

### SUMMARY OF THE INVENTION

The present invention provides efficient high-throughput methods and compositions for screening and identifying peptides which modulate a biological process, e.g., a predetermined biological process, in an organism. The present invention provides several advantages over existing approaches. For example, peptide libraries can be screened for the ability to inhibit a process on the biological level, such as the cellular or organismal level, without a need to know the mechanism of action at the molecular level. This is especially advantageous in the study of a complex and highly diverse disease such as, for example, cancer. Further, by working backward from a biologically, cellularly or organismally active peptide, the biomolecule targeted by the peptide can be identified, thereby validating the biomolecule as a therapeutic target in the process of interest.

Accordingly, the present invention provides a method of identifying a peptide which modulates a biological process, e.g., apoptosis, necrosis, protein trafficking, cell adhesion, membrane transport, cell motility, cell differentiation, infection, replication of a pathogenic organism, or the progression of a disease state. The method includes (a) contacting an organism (e.g., a pathogenic organism), a cell or a tissue with a peptide library comprising a multiplicity of peptides, wherein the peptides are fragments of at least one gene product of an organism; (b) assessing the ability of the peptides to modulate the biological process in the organism, the cell or the tissue; and (c) determining the amino acid sequence of at least one peptide shown in step (b) to modulate the biological process, thereby identifying the peptide as a modulator of the biological process. When a multiplicity of cells or a tissue is contacted with the peptide library, the method can, optionally, further include the step of contacting the cells or tissue with a pathogenic organism, such as a bacterium, virus, fungus or protozoan. In this embodiment the biological process to be assessed is infectivity or replication of the pathogenic organism

In one embodiment, the peptide library comprises a multiplicity of nested fragments of at least one gene product of an organism. For example, the nesting overlap may be 1, 2, 3, 4, 5, 6, 7, 8, 9, 10 or more amino acid residues. The peptides may each comprise about 50, 45, 40, 35, 30, 25, 20, 15, 10, 5 or less amino acid residues.

In another embodiment, the peptide library comprises a multiplicity of fragments of at least two, three, four, five or six gene products of an organism. In yet another embodiment, the peptide library may comprise a multiplicity of fragments of gene products from at least one, two, three, four, five or six chromosomes, or the entire genome of an organism.

In one embodiment, the cell, e.g., a mammalian cell such as a human cell, a yeast cell, an insect cell, or a plant cell, is derived from the same organism as the peptide library. In another embodiment, the tissue, e.g., a mammalian tissue, is derived from the same organism as the peptide library. In a further embodiment, the organism is the same organism as the organism from which the peptide library was derived.

In another embodiment, the ability of the peptides to modulate the biological process in the organism, the cell or the tissue is assessed by the use of immunohistochemistry, by monitoring a morphology change in the organism, the cell or the tissue, by measuring a change in expression of one or more genes or by measuring a change in levels of signal transduction, e.g., signal transduction that is primarily mediated by a G protein coupled receptor, in the organism, the cell or the tissue.

In one embodiment of the invention, the peptides may be fused to an additional amino acid sequence, such as a nuclear localization signal sequence, a membrane localization signal sequence, a farnesylation signal sequence, a transcriptional activation domain, or a transcriptional repression domain.

The methods of the invention may further include forming a second library comprising a multiplicity of peptide or non-peptide compounds designed based on the amino acid sequence identified in step (c) and selecting from the second library at least one peptide or non-peptide compound that modulates the biological process. In one embodiment, the peptides in the second library may consist of natural L-amino acids. In another embodiment, at least some of the peptides in the second library may comprise one or more non-natural amino acids, such as D-amino acids, β- or γ-amino acids or amino acids having a side chain which differs from any of the side chains of the twenty naturally occurring amino acids.

In another aspect, the present invention features peptides which modulate a biological process as identified by the methods of the invention, libraries containing these peptides, as well as pharmaceutical compositions comprising these peptides and pharmaceutically acceptable carriers.

In a further aspect, the invention provides the use of a peptide which modulates a biological process as identified by the methods of the invention, for the molecular modeling of a compound having similar binding or modulatory characteristics as the peptide.

In yet another aspect, the present invention features methods for treating a subject suffering from a disease or condition associated with an aberrant biological process, e.g., HIV infection or cancer, by administering to the subject a therapeutically effective amount of a peptide identified according to the methods of the invention.

In another aspect, the present invention provides kits for identifying a peptide which modulates a biological process, which include peptide libraries comprising a multiplicity of peptides, wherein the peptides are fragments of at least one gene product of an organism and instructions for use.

Other features and advantages of the invention will be apparent from the following detailed description and claims.

### DETAILED DESCRIPTION OF THE INVENTION

A wide variety of physiological processes proceed via a protein/protein interaction or a chain of two or more such interactions. Among these processes are those necessary for survival of and/or infection by pathogenic organisms and the development and/or maintenance of a number of disease states. A compound which is, for example, capable of inhibiting a protein/protein interaction essential for a disease process is potentially useful as a therapeutic agent for the treatment of a disease state, e.g., infection, cancer, inflammation, neurodegeneration or pain.

The present invention provides a method of identifying a peptide which modulates a biological process, e.g., apoptosis, necrosis, protein trafficking, cell adhesion, membrane transport, cell motility, cell differentiation, infection, replication of a pathogenic organism, or the progression of a disease state. The method includes (a) contacting an organism (e.g., a pathogenic organism), a cell or a tissue with a peptide library comprising a multiplicity of peptides, wherein the peptides are fragments of at least one gene product of an organism; (b) assessing the ability of the peptides to modulate the biological process in the organism, the cell or the tissue; and (c) determining the amino acid sequence of at least one peptide shown in step (b) to modulate the biological process, thereby identifying the peptide as a modulator of the biological process.

As used herein, the term “biological process” includes any biological process, for example, any molecular, cellular or organismal process. The biological process can be a molecular process, such as an enzymatic process, a protein/protein interaction, a protein/nucleic acid interaction, a nucleic acid/nucleic acid interaction, a peptide/protein interaction, or a protein/hormone interaction. The biological process can also be a cellular process, such as cell viability, protein expression, including expression of a particular protein; cell proliferation; cellular expression of one or more biomolecules; signal transduction; cell adhesion; cell differentiation; cell transformation; infectivity or apoptosis. The biological process may also be an organismal process, such as development or progression of a disease state or infection by a benign or pathogenic organism. The disease state can be a naturally occurring state or condition or a state or condition induced to mimic or resemble a naturally occurring disease state. For example, the biological process can be exhibited by any animal model of a disease or other undesirable medical condition.

As used herein, the term “organism” includes any living organism including animals, e.g., humans, mice, rats, monkeys, or rabbits; plants, e.g., Arabidopsis thaliana, rice, wheat, maize, tomato, alfalfa, oilseed rape, soybean, cotton, sunflower or canola; bacteria, e.g., Escherichia coli, Campylobacter, Listeria, Legionella, Staphylococcus, Streptococcus, Salmonella, Bordatella, Pneumococcus, Rhizobium, Chlamydia, Rickettsia, Streptomyces, Mycoplasma, Helicobacter pylori, Chlamydia pneumoniae, Coxiella burnetii, Bacillus Anthracis, and Neisseria; fungi, e.g., Rhizopus, neurospora, yeast, puccinia; Aspergillus, Blastomyces, Candida, Coccidioides, Cryptococcus, Epilermophyton, Hendersonula, Histoplasma, Microsporum, Paecilomyces, Paracoccidioides, Pneumocystis, Trichophyton, and Trichosporium; Protozoa: Plasmodium falciparum, Plasmodium vivax, Toxoplasma gondii, Trypanosoma rangeli, Trypanosoma cruzi, Cryptosporidum parvum, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Onchocerca volvulus, Theileria parva, Taenia hydatigena, Taenia ovis, Taenia saginata, Echinococcus granulosus and Mesocestoides corti parasites, such as tapeworms, e.g., Echinococcus granulosus, E. multilocularis, E. vogeli and E. oligarthrus; protozoa, e.g., Trypanosoma brucei. The term organism also includes viruses, e.g., human immunodeficiency virus, rhinoviruses, rotavirus, influenza virus, Ebola virus, simian immunodeficiency virus, feline leukemia virus, respiratory synctial virus, herpesvirus, pox virus, polio virus, parvoviruses, Kaposi's Sarcoma-Associated Herpesvirus (KSHV), adeno-associated virus (AAV), Sindbis virus, Lassa virus, West Nile virus, enteroviruses, such as 23 Coxsackie A viruses, 6 Coxsackie B viruses, and 28 echoviruses, Epstein-Barr virus, caliciviruses, astroviruses, and Norwalk virus; orbiviruses, orthoreoviruses, filoviruses, rabies virus, coronaviruses, bunyaviruses, arenaviruses, mumps virus, measles virus, parainfluenza virus, rubella virus, flaviviruses, alfaviruses, cytomega ovirus, HHV-6, HHV-7, adenovirus, hepatitis B virus, hepatitis C virus, hepatitis A virus, papillomavirus, jc virus, enteroviruses, and others as described in Field's Virology.

The term “cell” as used herein, includes any prokaryotic or eukaryotic cell. Examples of cells that may be used in the methods of the invention include fungal cells (i.e., yeast cells); insect cells (e.g., Schneider and sF9 cells); somatic or germ line mammalian cells; mammalian cell lines, e.g., HeLa cells (human), NIH3T3 (murine), RK13 (rabbit) cells, embryonic stem cells (e.g., D3 and J1); and mammalian cell types such as hematopoietic stem cells, myoblasts, hepatocytes, lymphocytes, and epithelial cells.

As used herein, the term “tissue” includes a group of similar cells and their intercellular substance joined together to perform a specific function. The term tissue includes any tissue of an organism, for example, epithelial tissue, connective tissue, muscle tissue, nervous tissue, vascular tissue, or osseous tissue.

As used herein, the term “peptide library” includes a collection of peptides which are fragments of at least one genome-encoded protein. The peptide library may include fragments of 2, 3, 4, 5, 6, 7, 8, 9, 10 or more proteins encoded by the same genome. The peptide library can also include fragments of 2-1000, 2-900, 2-800, 2-700, 2-600, 2-500, 2-400, 2-300, 2-200, 2-100 or 2-50 proteins encoded by the same genome. Preferably, the fragments, in aggregate, include all the amino acid residues of the encoded sequence. That is, for example, if the genome-encoded sequence consists of 200 amino acid residues, each of these residues is found in at least one peptide in the library, for example, bonded to at least one of the residues which are immediately adjacent in the intact encoded sequence. Such a library is said to be a “complete” library with respect to a specific genome-encoded protein. The peptide library can include fragments of a particular genome-encoded amino acid sequence which are contiguous, nested or a combination thereof. Fragments are contiguous when, if aligned end to end in the correct order, they reproduce the sequence of the genome-encoded amino acid sequence, that is, there is no overlap of the fragment sequences.

A “nested peptide library”, as the term is used herein, refers to a collection of peptides which includes fragments of one or more genome-encoded peptides where the N-terminus of at least some peptides overlaps by one or more amino acid residues with the C-terminus of at least one other peptide, and the C-terminus of at least some peptides overlaps by one or more amino acid residues with the N-terminus of at least one other peptide. The number of overlapping residues at each of the C- and N-termini is referred to as the degree of overlap or nesting overlap, and will typically be from 1 to n−1, where n represents the number of amino acid residues in the peptide. A library which consists of peptides having contiguous sequences has a degree of overlap of 0. The fragments in the library can have varying degrees of overlap across the intact genome-encoded sequence. That is, fragments of one or more particular regions of the intact sequence can have a degree of overlap which differs from that of fragments of another particular region of the intact sequence.

Various aspects of the invention are described further in the following subsections.

I. Methods of the Invention

The present invention provides a method of identifying a peptide which modulates a biological process, e.g., apoptosis, protein trafficking, cell adhesion, membrane transport, cell motility, cell differentiation, or the progression of a disease state. The method includes (a) contacting an organism (e.g., a pathogenic organism), a cell or a tissue with a peptide library comprising a multiplicity of peptides, wherein the peptides are fragments of at least one gene product of an organism; (b) assessing the ability of the peptides to modulate the biological process in the organism, the cell or the tissue; and (c) determining the amino acid sequence of at least one peptide shown in step (b) to modulate the biological process, thereby identifying the peptide as a modulator of the biological process.

In one non-limiting example, the library comprises 20 mers which are fragments of one or more genome-encoded proteins. In this embodiment, the peptides are contiguous or nested, with a degree of overlap typically ranging from 0 to about 10. Preferably, the sequences are nested with a constant degree of overlap. In general, the size and complexity of the library increases with an increasing degree of overlap. Also, the nested fragments can be produced starting from the amino terminus or the carboxy sequence of the intact sequence as in most cases a different set of peptides results from these starting points. The library can include the nested fragments resulting from starting at both the amino-terminus and the carboxy-terminus. For a genome-encoded protein comprising a single chain of 100 amino acid residues, one set of contiguous 20 mers will have the following sequences: 1-20; 21-40; 41-60; 61-80; and 81-100, for a total of 5 distinct sequences. If the degree of overlap is 2, one set of sequences beginning at the N-terminus would be 1-20; 18-37; 35-54; 52-71; 69-88; and 81-100. Beginning at the C-terminus, the sequences would be 81-100; 63-82; 45-64; 27-46; 9-28 and 1-20. Thus a total of 10 distinct sequences result from nesting with a degree of overlap of 2. If the degree of overlap is 5, the sequences beginning at the N-terminus would be 1-20; 16-35; 31-50; 46-65; 61-80; 76-95 and 80-100. Beginning at the C-terminus, the sequences would be 80-100; 65-84; 50-69; 35-54; 20-39; 5-24 and 1-20. Thus, a total of 12 distinct sequences result from a degree of overlap of 5. If the degree of overlap is 10, beginning at the N-terminus, the fragments produced are 1-20; 11-30; 21-40; 31-50; 41-60; 51-70; 61-80; 71-90; and 81-100, a total of 9 distinct sequences. If the degree of overlap is 19 (n-1), the possible peptides, starting from the N-terminus, include 1-20; 2-21; 3-22, 4-23; 5-24, and so forth, up to 81-100, for a total of 81 peptides.

The biological process of interest can be any biological process, for example, any molecular, cellular or organismal process. For example, the biological process can be a molecular process, such as an enzymatic process, a protein/protein interaction, a protein/nucleic acid interaction, a nucleic acid/nucleic acid interaction, a peptide/protein interaction, or a protein/hormone interaction. Optionally, the peptide library is initially screened for members which bind to a molecular target, such as a protein. Members that bind to the target can then be evaluated in a functional screen, which examines the functional consequences of binding to the target on a biological process, such as a molecular, cellular or organismal process. The biological process can also be a cellular process, such as cell viability, protein expression, including expression of a particular protein; cell proliferation; cellular expression of one or more biomolecules; signal transduction; cell adhesion; cell differentiation; cell transformation; infectivity or apoptosis. In another embodiment, the biological process is an organismal process, such as development or progression of a disease state or infection by a benign or pathogenic organism. The disease state can be a naturally occurring state or condition or a state or condition induced to mimic or resemble a naturally occurring disease state. For example, the biological process can be exhibited by any animal model of a disease or other undesirable medical condition.

In one embodiment, the ability of the peptide or peptides to modulate the biological activity of interest is assessed in an appropriate in vitro or in vivo assay or model. The in vitro assay can be a cell-free assay or a cell-based assay.

In one embodiment, the biological process is a protein/ligand interaction. In this embodiment, the library can be screened by contacting the library, either in its entirety or in fractions, with a first biomolecule, such as a protein, which is known or believed to be involved in the biological process of interest. Members of the library which bind the first biomolecule can, optionally, be eluted with a specific eluting agent, such as a second biomolecule, which is a binding partner of the first biomolecule. A member or members of the library which are found to bind the first biomolecule can then be evaluated in a functional screen, such as a cell based assay or an in vivo model.

In a first preferred embodiment, the peptide library is assessed in a cell-based assay. For example, cultured cells can be contacted with the peptide library, either as a peptide mixture comprising all of the library members, one or more sub-libraries, each including a subset of the library members, or as single peptides. The assay will, preferably, have a read-out that provides a quantitative or qualitative indication of the extent of modulation of the biological process of interest. Preferably, the library is assessed as a set of sub-libraries. A sub-library which exhibits activity in the assay can then be subdivided further, with the activity of each sub-sub-library in the assay determined. Subdivision of the library can continue until one or more peptides which individually exhibit activity in the assay are identified.

The present method is advantageously employed when the biological process of interest is a cell-based or organismal process, and is particularly effective when the process proceeds via one or more protein/protein interactions. However, a particularly important advantage of the method is that it does not require any knowledge of the mechanistic details of the process. The invention relates to the recognition that, in general, at least one peptide which will inhibit a protein/protein mediated process in an organism is encoded in the genome of that organism, such as a fragment of a genome-encoded protein. In one example, a peptide which inhibits the interaction of protein A with protein B can be a fragment of protein A which represents the domain of protein A which binds protein B. Alternatively, the peptide which inhibits the binding of protein A and protein B can be a fragment of protein B, for example, the domain of protein B which interacts with Protein A. It is expected that in many cases the peptide which is identified will include a portion of one of the protein partners. However, in any given case other peptide sequences unrelated to either of the protein partners may also be identified via the present method.

The inventive method also enables the identification and validation of potential biomolecular targets for a given disease state. For example, in one embodiment, the amino acid sequence of a peptide which is identified in step (c) as a modulator of a particular biological process can be compared to published amino acid and gene sequence data for the genome from which the library was derived or a related genome, thereby identifying one or more parent proteins encoded by the genome which can be fragmented to provide the identified peptide. The parent protein is then identified as a participant in the disease process and can be cloned and used as a target in conventional drug screening assays. A peptide identified by the present method can also be used to identify its target protein. For example, using pull-down techniques or other affinity selection techniques, the peptide can be used to separate its target protein from the cell's component proteins. The target protein is thus identified as a participant in the biological process of interest and can also be used as a molecular target in conventional drug screening assays, such as high throughput assays.

In one embodiment, at least some of the peptides within the library are fused to a peptide sequence which facilitates transport across the cell membrane. A variety of such membrane-permeable sequences are known and include sequences which are predominately hydrophobic, such as the signal sequence of Kaposi FGF, and others which include basic residues, such as sequences derived from the HIV TAT protein, antennapedia homeodomain, gelsolin and others. The genome-derived peptides in the library can be fused to a membrane-permeable sequence at the N-terminus or C-terminus. Suitable membrane-permeable sequences are described in U.S. Pat. Nos. 5,807,746; 6,043,339; 5,783,662; 5,888,762; 6,080,724; 5,670,617; 5,747,641; 5,804,604; WO 00/29427 and WO 99/29721, the contents of each of which are hereby incorporated by reference in their entirety.

The genome-encoded peptide libraries can be prepared via a variety of methods known in the art. For example, intact proteins can be fragmented, for example using a single protease or a combination of two or more proteases (e.g., trypsin, chymotrypsin or papain). This can result in random protein cleavage or protein cleavage at specific sequences, depending on the proteases used. The peptides can also be prepared using an expression library derived from the genome of interest. Such an expression library will comprise a library of vectors which include a nucleic acid sequence encoding a peptide which is a fragment of a protein encoded by the genome. Such expression libraries can be prepared using, for example, fragmented genomic DNA or synthetic nucleic acid sequences, for example, sequences derived from the genome of the organism and designed to provide peptides having the desired nesting and representing the entire genome or a desired portion of the genome. The expression libraries can also be prepared using fragmented cDNA, for example, cDNA prepared using cellular RNA transcripts and fragmented randomly, for example, using free radical methods (Fenton's reagent) or a collection of two or more nucleases or at specified positions using one or more nucleases. A collection of host cells, for example, bacterial cells, can be transfected with the expression library and the peptides expressed by the cells can be isolated using standard procedures.

The peptide libraries may also be prepared by any suitable method for peptide synthesis (stepwise or convergent), including solution-phase and solid-phase (bead or membrane base solid phase) chemical synthesis, or a combination of these approaches. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers are commercially available. Exemplary chemical syntheses of peptide libraries include the pin method (see, e.g., Geysen, H. M. et al. (1984) Proc. Natl. Acad. Sci. USA 81:3998-4002); the tea-bag method (see, e.g., Houghten, R. A. et al. (1985) Proc. Natl. Acad. Sci. USA 82:5131-5135); coupling of amino acid mixtures (see, e.g., Tjoeng, F. S. et al. (1990) Int. J. Pept. Protein Res. 35:141-146; U.S. Pat. No. 5,010,175 to Rutter et al.); and synthesis of spatial arrays of compounds (see, e.g., Fodor, S. P. A. et al. (1991) Science 251:767). In one embodiment, the peptide library is synthesized according to methods described in U.S. Pat. No. 6,040,423, the contents of which are hereby incorporated by reference in their entirety. The amino acid sequences of the peptides can be designed, for example, based on known or available genome information, for example, using the hypothetical translated amino acid sequences encoded by open reading frames in the genome.

In one embodiment, the peptide library comprises fragments of at least one protein encoded by the genome of a multicellular organism. The multicellular organism is preferably, a mammal or a domesticated non-mammalian animal, such as a chicken or turkey. Preferably, the multicellular animal is a mouse, a rat, a sheep, a cow, a pig, a dog, a cat or a goat, and more preferably, the multicellular organism is a primate, such as a monkey, an ape or a human. The library can include fragments of one or more genome-encoded proteins, as discussed above, and can also include proteins encoded by the genes on 1, 2, 3, 4, 5, 6, 7, 8, 9, 10 or more chromosomes.

In one embodiment, the peptide library comprises fragments of at least one protein which is encoded by a viral genome. Preferably, the library comprises fragments of two or more proteins encoded by the viral genome and, more preferably, is complete with respect to each of the encoded proteins. The proteins represented in the library can include structural proteins and non-structural proteins. In one embodiment, the library comprises fragments of each of the proteins encoded by the viral genome and, preferably, is complete with respect to each of the encoded proteins. In one embodiment, the peptide library comprises fragments of each protein encoded by the viral genome and, preferably, is complete with respect to each of the encoded proteins. The virus is, preferably, a pathogenic virus in mammals, such as humans. Suitable viruses include human immunodeficiency virus, rhinoviruses, rotavirus, influenza virus, Ebola virus, simian immunodeficiency virus, feline leukemia virus, respiratory syncytial virus, herpesvirus, pox virus, polio virus, parvoviruses, Kaposi's Sarcoma-Associated Herpesvirus (KSHV), adeno-associated virus (AAV), Sindbis virus, Lassa virus, West Nile virus, enteroviruses, such as 23 Coxsackie A viruses, 6 Coxsackie B viruses, and 28 echoviruses, Epstein-Barr virus, caliciviruses, astroviruses, and Norwalk virus. Virus may, for example, be harvested from an infected cell supernatant or infected organism, the virions may then be purified, and the proteins may be proteolytically digested to generate the peptide library.

In this embodiment, the peptide library is assessed for the ability to modulate, preferably inhibit, a process associated with the ability of the virus to infect a host cell, use the host cell for the production of viral proteins and/or replicate within the host cell. Thus, the assay can involve contacting potential host cells with the peptide library or sub-library in the presence of the virus, and assessing the ability of the library to inhibit viral entry, viral protein production or viral replication. Such assays are known in the art.

In another embodiment, the peptides are fragments of one or more proteins encoded by a bacterial genome. Preferably, the library comprises fragments of two or more proteins encoded by the bacterial genome and, more preferably, is complete with respect to each of the encoded proteins. In one embodiment, the library comprises fragments of each of the proteins encoded by the bacterial genome and, preferably, is complete with respect to each of the encoded proteins. The bacterium is, preferably, a pathogenic bacterium in mammals, such as humans, although under certain circumstances, a non-pathogenic strain having significant genomic similarity to a pathogenic strain can be used. Such bacteria are known in the art and include pathogenic strains of E. coli, Campylobacter, Listeria, Legionella, Staphylococcus, Streptococcus, Salmonella, Bordetella, Pneumococcus, Rhizobium, Chlamydia, Rickettsia, Streptomyces, Mycoplasma, Helicobacter pylori, Chlamydia pneumoniae, Coxiella burnetii, and Neisseria.

Genome sequences for various organisms are well known in the art. The sites where the genomic sequences of various representative organisms may be found are set forth in the following Table.

| ORGANISM | SITE OF SEQUENCE INFORMATION |
|---|---|
| MAMMALS: | |
| Mouse | http://www.informatics.jax.org/ |
| (Mus musculus) | |
| Rat | http://www.ncbi.nlmnih.gov/htbin- |
| (Rattus) | post/Taxonomy/wgetorg?mode=Info&id=10114& |
| | lvl=3&keep=l&srchmode=1&unlock |
| Human | http://www.ncbi.nlmnih.gov:80/cgi-bin/Entrez/ |
| (Homo Sapiens) | framik?db=Genome&gi=1 |
| Dog | http://www.ncbi.nlm.nih.gov:80/htbin- |
| (Canis Familiaris) | post/Taxonomy/wgetorg?mode=Info&id=9611& |
| | lvl=3&keep=1&srchmode=1&unlock |
| Sheep | http://www.ncbi.nlm.nih.gov:80/htbin- |
| (Ovis Aries) | post/Taxonomy/wgetorg?mode=Info&id=9940& |
| | lvl=3&keep=1&srchmode=1&unlock |
| Goat | http://www.ncbi.nlm.nih.gov:80/htbin- |
| (Capra Hircus) | post/Taxonomy/wgetorg?mode=Info&id=9922& |
| | lvl=3&keep=1&srchmode=1&unlock |
| Gorilla | http://www.ncbi.nlm.nih.gov:80/htbin- |
| (Gorilla Gorilla) | post/Taxonomy/wgetorg?mode=Info&id=9527& |
| | lvl=3&keep=1&srchmode=1&unlock |
| Monkey | http://www.ncbi.nlm.nih.gov:80/htbin- |
| (Cercopithecidae) | post/Taxonomy/wgetorg?mode=Info&id=9527& |
| | lvl=3&keep=1&srchmode=1&unlock |
| VIRUSES: | |
| Respiratoy | http://www.ncbi.nlm.nih.gov:80/cgi-bin/Entrez/ |
| Syncytial Virus | framik?db=Genome&gi=12176 |
| Herpesvirus | http://www.ncbi.nlm.nih.gov:80/cgi-bin/Entrez/ |
| (eg. Human) | framik?db=Genome&gi=12187 |
| Enterovirus (eg. #70) | http://www.ncbi.nlm.nih.gov:80/cgi-bin/Entrez/ |
| | framik?db=Genome&gi=11615 |
| Echovrus (eg #23) | http://www.ncbi.nlm.nih.gov:80/cgi-bin/Entrez/ |
| | framik?db=Genome&gi=13513 |
| Calicivirus | http://www.ncbi.nlm.nih.gov:80/cgi-bin/Entrez/ |
| (eg. Norwalk) | framik?db=Genome&gi=13999 |
| Astrovirus | http://www.ncbi.nlm.nih.gov:80/cgi-bin/Entrez/ |
| (eg. Human type 8) | framik?db=Genome&gi=15469 |
| Poliovirus | http://www.ncbi.nlm.nih.gov:80/cgi-bin/Entrez/ |
| (eg Human) | framik?db=Genome&gi=10328 |
| Coxsackie (eg B5) | http://www.ncbi.nlm.nih.gov:80/cgi-bin/Entrez/ |
| | framik?db=Genome&gi=10037 |
| Rhinovirus | http://www.ncbi.nlm.nih.gov:80/cgi-bin/Entrez/ |
| (eg Human type 14) | framik?db=Genome&gi=10274 |
| Human | http://www.ncbi.nlm.nih.gov:80/cgi-bin/Entrez/ |
| Immunodeficiency | framik?db=Genome&gi=12171 |
| Virus | |
| Simian | http://www.ncbi.nlm.nih.gov:80/cgi-bin/Entrez/ |
| Immunodeficiency | framik?db=Genome&gi=10371 |
| Virus | |
| Feline Leukemia Virus | http://www.ncbi.nlm.nih.gov:80/cgi-bin/Entrez/ |
| | framik?db=Genome&gi=13946 |
| Pox viruses | http://www.ncbi.nlm.nih.gov/entrez/guery.fcgi? |
| | db=Genome |
| Ebola Virus | http://www.ncbi.nlm.nih.gov/entrez/query.fcgi? |
| | db=Genome |
| Influenza Viruses | http://www.ncbi.nlm.nih.gov/entrez/guery.fcgi? |
| | db=Genome |
| Adeno-associated | http://www.ncbi.nlm.nih.gov/entrez/query.fcgi? |
| viruses | db=Genome |
| Sindbis virus | http://www.ncbi.nlm.nih.gov/entrez/query.fcgi? |
| | db=Genome |
| West Nile virus | http://www.ncbi.nlm.nih.gov/entrez/query.fcgi? |
| | db=Genome |
| Rabies | http://www.ncbi.nlm.nih.gov/entrez/query.fcgi? |
| | db=Taxonomy |
| Parvovirus | http://www.ncbi.nlm.nih.gov/entrez/query.fcgi? |
| | db=Taxonomy |
| BACTERIA: | |
| E. Coli | http://www.ncbi.nlm.nih.gov:80/PMGifs/ |
| | Genomes/micr.html |
| Campylobacter | http://www.ncbi.nlm.nih.gov:80/PMGifs/ |
| | Genomes/micr.html |
| Listeria | http://www.ncbi.nlm.nih.gov/entrez/query.fcgi? |
| | db=Nucleotide |
| Legionella | http://www.ncbi.nlm.nih.gov/entrez/query.fcgi? |
| | db=Nucleotide |
| Helicobacter Pylori | http://www.ncbi.nlm.nih.gov:80/PMGifs/ |
| | Genomes/micr.html |
| Neisseria | http://www.ncbi.nlm.nih.gov:80/PMGifs/ |
| | Genomes/micr.html |
| Mycobacterium | http://www.ncbi.nlm.nih.gov:80/PMGifs/ |
| | Genomes/micr.html |
| Salmonella | http://pedant.mips.biochem.mpg.de/cgi-bin/ |
| | wwwfly.pl?Set=Styphi&Page=index |
| Chlamydia | http://www.ncbi.nlm.nih.gov:80/PMGifs/ |
| | Genomes/micr.html |
| Vibrio Cholerae | http://www.ncbi.nlm.nih.gov:80/PMGifs/ |
| | Genomes/micr.html |
| Pyrococcus | http://www.ncbi.nlm.nih.gov:80/PMGifs/ |
| | Genomes/micr.html |
| Haemophilus | http://www.ncbi.nlm.nih.gov:80/PMGifs/ |
| | Genomes/micr.html |
| Rickettsia | http://www.ncbi.nlm.nih.gov:80/PMGifs/ |
| | Genomes/micr.html |
| Mycoplasma | http://www.ncbi.nlm.nih.gov:80/PMGifs/ |
| | Genomes/micr.html |
| Listeria | http://www.ncbi.nlm.nih.gov:80/htbin- |
| | post/Taxonomy/wgetorg?mode=Info&id=1637& |
| | lvl=3&keep=1&srchmode=1&unlock |
| | http://www.tigr.org/tdb/mdb/mdbinprogress.html |
| Legionella | http://www.ncbi.nlm.nih.gov:80/htbin- |
| | post/Taxonomy/wgetorg?mode=Info&id=445& |
| | lvl=3&keep=1&srchmode=1&unlock |
| | http://www.tigr.org/tdb/mdb/mdbinprogress.html |
| Staphylococcus | http://www.ncbi.nlm.nih.gov:80/htbin- |
| | post/Taxonomy/wgetorg?mode=Info&id=1279& |
| | lvl=3&keep=1&srchmode=1&unlock |
| | http://www.tigr.org/tdb/mdb/mdbinprogress.html |
| Streptococcus | http://www.ncbi.nlm.nih.gov:80/htbin- |
| | post/Taxonomy/wgetorg?mode=Info&id=1301& |
| | lvl=3&keep=1&srchmode=1&unlock |
| | http://www.tigr.org/tdb/mdb/mdbinprogress.html |
| Salmonella | http://www.ncbi.nlm.nih.gov:80/htbin- |
| | post/Taxonomy/wgetorg?mode=Info&id=590& |
| | lvl=3&keep=1&srchmode=1&unlock |
| | http://genome.wustl.edu/gsc/Projects/ |
| | bacteria.shtml |
| Bordetella | http://www.ncbi.nlm.nih.gov/entrez/query.fcgi? |
| | db=Nucleotide&cmd=Search&dopt= |
| | DocSum&term=txid517%5BOrganism%5D& |
| | button=Get+Sequences |
| Coxiella | http://www.ncbi.nlm.nih.gov:80/htbin- |
| | post/Taxonomy/wgetorg?mode=Info&id=776& |
| | lvl=3&keep=1&srchmode=1& unlock |
| Rotavirus | http://www.ncbi.nlm.nih.gov:80/htbin- |
| | post/Taxonomy/wgetorg?mode=Info&id=10912& |
| | lvl=3&keep=1&srchmode=1&unlock |
| Rhizobium | http://www.ncbi.nlm.nih.gov:80/htbin- |
| | post/Taxonomy/wgetorg?mode=Info&id=379& |
| | lvl=3&keep=1&srchmode=4&unlock |
| Streptomyces | http://www.ncbi.nlm.nih.gov:80/htbin- |
| | post/Taxonomy/wgetorg?mode=Info&id=1883& |
| | lvl=3&keep=1&srchmode=1&unlock |
| Epstein-Barr Virus | http://www.ncbi.nlm.nih.gov/cgi-bin/Entrez/ |
| | framik?db=Genome&gi=10040 |
| OTHER: | |
| Plasmodium | http://www.ncbi.nlm.nih.gov:80/PMGifs/ |
| | Genomes/euk.html |

In another embodiment, the invention relates to the use of expression libraries which encode a peptide library of the invention, as described above. Such expression libraries comprise a library of nucleic acid fragments contained as inserts in an expression library. Thus, an expression library comprises a library of vectors, each of which encodes a peptide which is a fragment of a protein encoded by the genome of an organism, such as a mammal, a bacterium or a virus. The nucleic acid fragments can be prepared by synthetic methods known in the art or, preferably, by fragmenting genomic DNA or cDNA. Genomic DNA and cDNA can be fragmented using one or more of a variety of nucleases as are known in the art or by random cleavage using, for example, Fenton's reagent. Preferably, the expression vectors encode a library of nested fragments of one or more genome-encoded proteins.

The expression libraries of the invention can be used in a method for identifying a peptide which modulates a biological process. The method includes (1) providing an expression library comprising expression vectors which each encode a peptide which is a fragment of a protein encoded by the genome of an organism; (2) transfecting cells with the expression library; (3) identifying one or more transfected cells in which the cellular process is modulated; (4) identifying the expression vector or expression vectors in the transfected cell or cells in which the cellular process is modulated; and (5) determining the amino acid sequence of the peptide or peptides encoded by the expression vector or vectors identified in step (4); thereby identifying a peptide which modulates the cellular process.

Vectors that may be used to express the peptide libraries of the invention include art known expression vectors, such as viral vectors (e.g., replication defective retroviruses, adenoviruses and adeno-associated viruses). The expression vectors typically include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences).

The recombinant expression vectors can be designed for expression of the peptide libraries in prokaryotic or eukaryotic cells. For example, the peptide libraries can be expressed in bacterial cells such as E. coli, insect cells (using baculovirus expression vectors), yeast cells, plant cells, avian cells, fungal cells or mammalian cells. Suitable host cells are discussed further in Goeddel, Gene Expression Technology: Methods in Enzymology 185, Academic Press, San Diego, Calif. (1990). Alternatively, the recombinant expression vectors can be transcribed and translated in vitro, for example using T7 promoter regulatory sequences and T7 polymerase.

Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.). Examples of vectors for expression in insect cells include the baculovirus expression vectors, e.g., the pAc series (Smith et al. (1983) Mol. Cell Biol. 3:2156-2165) and the pVL series (Lucklow and Summers (1989) Virology 170:31-39). Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

Vector DNA carrying the peptide libraries of the invention can be introduced into prokaryotic or eukaryotic cells via conventional transformation or transfection techniques, such as calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), as well as in U.S. Pat. No. 5,955,275, the contents of which are incorporated by reference.

II. Assays Used in the Methods of the Invention

The ability of the peptide or peptides to modulate the biological activity of interest is assessed in an appropriate in vitro or in vivo assay or model. The in vitro assay can be a cell-free assay or a cell-based assay.

Cells, tissues or whole organisms (e.g., the animal models described herein) may be contacted with a peptide library or transfected with a vector or multiplicity of vectors coding for the peptide library and the effects of the peptide library members on a biological process, e.g., apoptosis, protein trafficking, cell adhesion, membrane transport, cell motility, cell differentiation, or the progression of a disease state, can be detected as described herein.

For example, apoptotic cells may be identified using APOPTEST ™, TUNEL staining methods or other art known methods, both before and after the cells or tissues have been contacted with a peptide library. The APOPTEST ™ method utilizes an annexin V antibody to detect cell membrane re-configuration that is characteristic of cells undergoing apoptosis. Apoptotic cells stained in this manner can then sorted either by fluorescence activated cell sorting (FACS), or by adhesion and panning using immobilized annexin V antibodies.

A T cell hybridoma (3DO) which has been cross-linked with a T cell receptor to induce programmed cell death (as described in Ashwell J. D. et al. (1990) J. Immunol. 144:3326) may also be contacted with a peptide library or transfected with a vector or multiplicity of vectors coding for a peptide library of the invention. The effect of the peptide library members on programmed cell death can then be detected, e.g., by monitoring nuclear chromatin changes.

Cell motility in response to peptide library members may be detected by observing, for example, changes in actin filament assembly at the leading edge of the cell, changes in filament crosslinking, changes in actin network retrograde flow, changes in filament disassembly, changes in actin monomer sequestration, changes in monomer recycling and anterograde diffusion, and changes in anterograde organelle flow and lagging-edge retraction.

Whole organisms (or cells or tissues derived therefrom) may also be contacted with the peptide libraries or transfected with a vector or multiplicity of vectors coding for the peptide libraries of the invention. Suitable organisms include animal models for a disease state.

Animal models of cardiovascular disease that may be used in the methods of the invention include apoB or apoR deficient pigs (Rapacz, et al., 1986, Science 234:1573-1577); Watanabe heritable hyperlipidemic (WHHL) rabbits (Kita et al., 1987, Proc. Natl. Acad. Sci USA 84: 5928-5931); non-recombinant, non-genetic animal models of atherosclerosis such as, for example, pig, rabbit, or rat models in which the animal has been exposed to either chemical wounding through dietary supplementation of LDL, or mechanical wounding through balloon catheter angioplasty; rat myocardial infarction models (described in, for example, Schwarz, E R et al. (2000) J. Am. Coll. Cardiol. 35:1323-1330); and models of chromic cardiac ischemia in rabbits (described in, for example, Operschall, C et al. (2000) J. Appl. Physiol. 88:1438-1445).

Animal models of tumorigenesis that may be used in the methods of the invention are well known in the art (reviewed in Animal Models of Cancer Predisposition Syndromes, Hiai, H and Hino, 0 (eds.) 1999, Progress in Experimental Tumor Research, Vol. 35; Clarke A R Carcinogenesis (2000) 21:435-41) and include, for example, animals carrying carcinogen-induced tumors (Rithidech, K et al. Mutat Res (1999) 428:33-39; Miller, M L et al. Environ Mol Mutagen (2000) 35:319-327); animals in which tumor cells have been injected and/or transplanted; and animals bearing mutations in growth regulatory genes, for example, oncogenes (e.g., ras) (Arbeit, J M et al. Am J Pathol (1993) 142:1187-1197; Sinn, E et al. Cell (1987) 49:465-475; Thorgeirsson, S S et al. Toxicol Lett (2000) 112-113:553-555) and tumor suppressor genes (e.g., p53) (Vooijs, M et al. Oncogene (1999) 18:5293-5303; Clark A R Cancer Metast Rev (1995) 14:125-148; Kumar, T R et al. J Intern Med (1995) 238:233-238; Donehower, L A et al. (1992) Nature 356215-221). Furthermore, experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000) 462:219-226; Bostwick, D G et al. Prostate (2000) 43:286-294).

Models for studying angiogenesis in vivo include tumor cell-induced angiogenesis and tumor metastasis (Hoffman, R. M. (1998-99) Cancer Metastasis Rev. 17:271-277; Holash, J. et al. (1999) Oncogene 18:5356-5362; Li, C. Y. et al. (2000) J. Natl Cancer Inst. 92:143-147), matrix induced angiogenesis (U.S. Pat. No. 5,382,514), the disc angiogenesis system (Kowalski, J. et al. (1992) Exp. Mol. Pathol. 56:1-19), the rodent mesenteric-window angiogenesis assay (Norrby, K (1992) EXS 61:282-286), experimental choroidal neovascularization in the rat (Shen, W Y et al. (1998) Br. J. Ophthalmol. 82:1063-1071), and the chick embryo development (Brooks, P C et al. Methods Mol. Biol. (1999) 129:257-269) and chick embryo chorioallantoic membrane (CAM) models (McNatt L G et al. (1999) J. Ocul. Pharmacol. Ther. 15:413-423; Ribatti, D et al. (1996) Int. J. Dev. Biol. 40:1189-1197), and are reviewed in Ribatti, D and Vacca, A (1999) Int. J. Biol. Markers 14:207-213.

Models for studying vascular tone in vivo include the rabbit femoral artery model (Luo et al. (2000) J. Clin. Invest. 106:493-499), eNOS knockout mice (Hannan et al. (2000) J. Surg. Res. 93:127-132), rat models of cerebral ischemia (Cipolla et al. (2000) Stroke 31:940-945), the renin-angiotensin mouse system (Cvetkovik et al. (2000) Kidney Int. 57:863-874), the rat lung transplant model (Suda et al. (2000) J. Thorac. Cardiovasc. Surg. 119:297-304), the New Zeland White rabbit model of intracranial hypertension (Richards et al. (1999) Acta Neurochir. 141:1221-1227), the spontaneously hypertensive (SH) rat neurogenic model of chronic hypertension (Stekiel et al. (1999) Anesthesiology 91:207-214), the Prague hypertensive rat (PHR) (Vogel et al. (1999) Clin. Sci. 97:91-98), chronically angiotensin II (Ang II)-infused rats (Pasquie et al. (1999) Hypertension 33:830-834), Dahl-salt-sensitive rats (Boulanger (1999) J. Mol. Cell. Cardiol. 31:39-49), the mouse model of arterial remodeling (Bryant et al. (1999) Circ. Res. 84:323-328), and the obese Zucker (fa/fa) rat (Golub et al. (1998) Hypertens. Res. 21:283-288).

In another embodiment, the peptide library is assessed for the ability to induce a cellular second messenger (e.g., intracellular Ca²⁺, diacylglycerol, IP₃), for the ability to induce a reporter gene (comprising a target-responsive regulatory element operatively linked to a nucleic acid encoding a detectable marker, e.g., chloramphenicol acetyl transferase), or the ability to phosphorylate an intracellular substrate. The ability of a peptide library member to phosphorylate an intracellular substrate can be determined by, for example, an in vitro kinase assay. Briefly, a cell of interest can be incubated with the peptide library (or sub-library) or transfected with a vector or mulptiplicity of vectors coding for the peptide library (or sub-library) and radioactive ATP, e.g., [γ-³²P] ATP, in a buffer containing MgCl₂ and MnCl₂, e.g., 10 mM MgCl₂ and 5 mM MnCl₂. Following the incubation, the cellular components can be separated by SDS-polyacrylamide gel electrophoresis under reducing conditions, transferred to a membrane, e.g., a PVDF membrane, and autoradiographed. The appearance of detectable bands on the autoradiograph indicates that cellular substrates have been phosphorylated. Phosphoaminoacid analysis of the phosphorylated substrate can also be performed in order to determine which residues on the substrate are phosphorylated. Briefly, the radiophosphorylated protein band can be excised from the SDS gel and subjected to partial acid hydrolysis. The products can then be separated by one-dimensional electrophoresis and analyzed on, for example, a phosphoimager and compared to ninhydrin-stained phosphoaminoacid standards.

In another embodiment, the peptide library is assessed for the ability to modulate, preferably inhibit, a process associated with the ability of the virus to infect a host cell, use the host cell for the production of viral proteins and/or replicate within the host cell. Thus, the assay can involve contacting potential host cells with the peptide library or sub-library or transfecting potential host cells with a vector or multiplicity of vectors coding for the peptide library in the presence of the virus, and assessing the ability of the library to inhibit viral entry, viral protein production or viral replication. Such assays are known in the art and include those described in, for example, “General Viral Experiments” Ed. by Fellow Membership of The National Institute of Health, Maruzen Co., Ltd. (1973); U.S. Pat. Nos. 6,140,063; 6,140,063; 6,087,094; 6,071,744; 5,843,736; and 5,565,425, the contents of each of which are incorporated herein by reference.

In yet another embodiment, the peptide library is assessed for the ability to modulate, preferably inhibit, a process associated with the ability of a bacterium to infect a host cell. Assays that may be used for this purpose include, but are not limited to, those described in U.S. Pat. Nos. 5,654,141 and 6,165,736, the contents of each of which are incorporated herein by reference.

Several assays that may be used in the methods of the invention, including assays that measure the expression of the BRCA1 gene or genes in the p53 and p21 pathways and assays that measure cell contact inhibition, are described in, for example, U.S. Pat. No. 5,998,136, the entire contents of which are incorporated herein by reference.

III. Drug Development

Another embodiment of the invention includes the use of the peptides identified in the methods of the invention as being modulators of a biological process, as lead molecules for drug development. For example, using any art recognized molecular modeling techniques (e.g., the STR3DI MOLECULAR MODELER available by Exorga, Inc.) a peptide identified in the methods of the invention can be used to design and synthesize other molecules having the desirable function of the peptide but also having other desirable traits such as improved plasma half-life, improved solubility, and improved potency.

This invention is further illustrated by the following examples which should not be construed as limiting. The contents of all references, patents and published patent applications cited throughout this application, as well as the Figures and the Sequence Listing are hereby incorporated by reference.

### EXAMPLES

### Example 1

Preparation of a Human Genomic DNA Expression Vector

Human genomic DNA was digested with a combination of restriction enzymes (AciI, Hinp1I, HpaII, HpyCH4IV BfaI, MseI, NlaIII, RsaI, Sau3AI). The ends of the DNA fragments were made blunt by incubation with Klenow enzyme and deoxynucleotides. The pCLNCX retroviral vector, which had previously been modified to contain XhoI and Not I restriction sites between the existing HindIII and ClaI restriction sites, was further modified by insertion of the following oligonucleotides into the XloI/NotI sites:

| | |
|---|---|
| XhoI kozak``PmII````NotI | |
| TCGAGCCACCATGCACGTGGTAGCTAGCTAGC | (SEQ ID NO:1) |
| CGGTGGTACGTGCACCATCGATCGATCGCCGG | (SEQ ID NO:2) |
| TCGAGCCACCATGGCACGTGGTAGCTAGCTAGC | (SEQ ID NO:3) |
| CGGTGGTACCGTGCACCATCGATCGATCGCCGG | (SEQ ID NO:4) |
| TCGAGCCACCATGGGCACGTGGTAGCTAGCTAGC | (SEQ ID NO:5) |
| CGGTGGTACCCGTGCACCATCGATCGATCGCCGG | (SEQ ID NO:6) |

The insertion of these oligonucleotides provided a kozak sequence, an ATG start codon, and a PmII restriction site for cloning the blunt ended genomic DNA fragments in all three reading frames.

The pCLNCX vector containing the genomic DNA fragment library was packaged by co-transfection into COS cells with a vector encoding moloney leukemia virus gag and pol proteins, and a vector encoding the vesicular stomatitis virus envelope glycoprotein.

### Example 2

Identification of Viral Peptides that Interfere with the TNFα Signaling Pathway

The virus collected the from the COS supernatant of Example 1 seventy-two hours post transfection is used to infect MCF-7N breast cancer cells. Twenty four hours post infection, MCF-7N cells are treated with TNFα to induce apoptosis. Surviving colonies are collected after 7 days and expanded. RNA collected from the surviving clones is used as a PCR template to amplify the genomic DNA fragments that interfere with the TNFα signaling pathway, thus promoting cell survival. The genomic DNA fragments are then sequenced and identified by searching Genebank™.

### Example 3

Identification of Viral Peptides that Interfere with the Androgen Signaling Pathway

The virus collected from the COS supernatant of Example 1 seventy-two hours post transfection is used to infect MDA PCA 2b prostate cancer cells stably transfected with EGFP under the control of the prostate-specific-antigen promoter. These cells express EGFP only when dihydrotestosterone is included in the culture medium. Four days post infection, cells with reduced expression of EGFP are selected by cell sorting. RNA collected from the surviving clones is used as a PCR template to amplify the genomic DNA fragments responsible for interference with the androgen signaling pathway. The genomic DNA fragments are sequenced and identified by searching Genebank™.

### Example 4

Identification of Viral Peptides that Modulate Influenza Virus Pathology

Oligonucleotides encoding peptides spanning 20 amino acid stretches of all open reading frame of influenza with 10 amino acid overlaps are synthesized, amplified by PCR and inserted into a retroviral vector that contains the selectable drug marker neomycin resistance. MDCK cells are then infected with the retrovirus encoding the library of overlapping peptides and plated one cell per well in 96 well tissue culture plates. The cells are allowed to grow in the presence of neomycin. Once the cells are 60-80% confluent, media is replaced with media not containing neomycin and cells are infected with an m.o.i. of 1 with either a recombinant influenza virus encoding luciferase or wild-type influenza virus. In the first case, wells are analyzed for the expression levels of luciferase twenty four hours post infection and compared to the levels of luciferase from infections of equal numbers of cells that were infected with retroviruses containing irrelevant peptide coding regions. In the second case, wells are analyzed for the extent of viral cytopathic effect two to three days post-infection. DNA is then extracted from wells that showed less luciferase activity or CPE and PCR is used to amplify the peptide coding regions of the retrovirus. This PCR fragment is sequenced to identify the viral peptide that inhibited influenza pathology in vitro, inserted into a new retroviral vector and assayed again in the same assay. If the repeated assay again shows that expression of the viral peptide inhibited influenza pathology as assessed by reduced luciferase activity or CPE, the DNA is isolated again, PCR amplified and sequenced to confirm that the sequence is the same as the DNA obtained from the first assay. If the two DNA sequences are the same, then peptides corresponding to the inhibitory sequences are synthesized either with or without a membrane permeable sequence. These peptides are then added to MDCK cells in various concentrations, including a mock control, followed by infection of the cells with the recombinant influenza virus encoding luciferase or wild-type influenza. Twenty four hours later, the wells are assayed for luciferase activity or two to three days later the cells are assessed for CPE. Peptides that show inhibition of luciferase activity or CPE compared to mock controls are further analyzed and optimized as potential therapeutics for influenza virus infection.

This same protocol can be accomplished using retroviral vectors containing CDNA isolated from virally infected cells, sheared or restriction enzyme cleaved viral genomic DNA or by using synthesized peptides directly that cover some or all of the open reading frames contained in a viral genome.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method of identifying a peptide which modulates a biological process, comprising:
(a) contacting an organism, a cell or a tissue with a peptide library comprising a multiplicity of peptides, wherein said peptides are fragments of at least one gene product of an organism;
(b) assessing the ability of said peptides to modulate the biological process in said organism, said cell or said tissue; and
(c) determining the amino acid sequence of at least one peptide shown in step (b) to modulate the biological process, thereby identifying the peptide as a modulator of the biological process.

2. The method of claim 1, wherein the biological process is apoptosis.

3. The method of claim 1, wherein the biological process is protein trafficking.

4. The method of claim 1, wherein the biological process is cell adhesion.

5. The method of claim 1, wherein the biological process is membrane transport.

6. The method of claim 1, wherein the biological process is cell motility.

7. The method of claim 1, wherein the biological process is cell differentiation.

8. The method of claim 1, wherein the biological process is the progression of a disease state.

9. The method of claim 1, wherein the organism is a pathogenic organism.

10. The method of claim 1, wherein the peptide library comprises a multiplicity of nested fragments of at least one gene product of the organism.

11. The method of claim 10, wherein the peptides each comprise 10 or more amino acid residues and the nesting overlap is 1 or more amino acid residues.

12. The method of claim 11, wherein the nesting overlap is from 1 to 5 amino acid residues.

13. The method of claim 1, wherein the peptide library comprises a multiplicity of fragments of at least two gene products of the organism.

14. The method of claim 1, wherein the peptide library comprises a multiplicity of fragments of gene products from at least one chromosome of the organism.

15. The method of claim 1, wherein the peptides each comprise about 50 or less amino acid residues.

16. The method of claim 1, wherein the peptides each comprise about 30 or less amino acid residues.

17. The method of claim 1, wherein the peptides each comprise about 20 or less amino acid residues.

18. The method of claim 1, wherein the peptides each comprise about 5 or less amino acid residues.

19. The method of claim 1, wherein said cell or said tissue is derived from said organism.

20. The method of claim 1, wherein said cell is a mammalian cell.

21. The method of claim 20, wherein said mammalian cell is a human cell.

22. The method of claim 1, wherein said cell is a yeast cell.

23. The method of claim 1, wherein said cell is an insect cell.

24. The method of claim 1, wherein said cell is a plant cell.

25. The method of claim 1, wherein the ability of said peptides to modulate the biological process in said organism, said cell or said tissue is assessed using immunohistochemistry.

26. The method of claim 1, wherein the ability of said peptides to modulate the biological process in said organism, said cell or said tissue is assessed by monitoring a morphology change in said organism, said cell or said tissue.

27. The method of claim 1, wherein the ability of said peptides to modulate the biological process in said organism, said cell or said tissue is assessed by measuring a change in levels of signal transduction in said organism, said cell or said tissue.

28. The method of claim 27, wherein the change in levels of signal transduction is primarily mediated by a G protein coupled receptor.

29. The method of claim 1, wherein said peptides are fused to an additional amino acid sequence selected from the group consisting of a nuclear localization signal sequence, a membrane localization signal sequence, a farnesylation signal sequence, a transcriptional activation domain, and a transcriptional repression domain.

30. The method of claim 1, further comprising forming a second library comprising a multiplicity of peptide or non-peptide compounds designed based on the amino acid sequence identified in step (c) and selecting from the second library at least one peptide or non-peptide compound that modulates the biological process.

31. A method for identifying a peptide which modulates a biological process, comprising:
(a) providing a library of expression vectors, each of said vectors comprising a nucleic acid sequence which encodes a member of a peptide library, wherein the peptide library comprises fragments of one or more proteins which are encoded by the genome of an organism;
(b) contacting a multiplicity of cells with the library of expression vectors under conditions suitable for transfection of the cells by the expression vectors and expression of the encoded peptide library within the cells;
(c) selecting a cell in which the biological process is modulated; and
(d) determining the nucleic acid sequence of step (a) in the cell of step (c), wherein the peptide which is encoded by the nucleic acid sequence is identified as a peptide which modulates the biological process.

32. The method of claim 31, wherein the library of expression vectors comprises viral vectors.

33. The method of claim 31, wherein each vector further includes a regulatory sequence which is operatively linked to the nucleic acid sequence which encodes a member of a peptide library.

34. The method of claim 31, where the peptide library comprises fragments of two or more proteins encoded by the genome of an organism.

35. The method of claim 34, wherein the peptide library comprises fragments of five or more proteins encoded by the genome of an organism.

36. The method of claim 35, wherein the peptide library comprises fragments of ten or more proteins encoded by the genome of an organism.

37. The method of claim 36, wherein the peptide library comprises fragments of fifteen or more proteins encoded by the genome of an organism.

38. The method of claim 37, wherein the peptide library comprises fragments of twenty or more proteins encoded by the genome of an organism.

39. The method of claim 35, wherein the peptide library comprises fragments of twenty-five or more proteins encoded by the genome of an organism

40. The method of claim 34, wherein the peptide library comprises fragments of each protein encoded by the genome of an organism.

41. The method of claim 31, wherein the cells are derived from the organism.

42. The method of claim 41, wherein the organism is a mammal, an avian animal, a bacterium, a fungus or a protozoan.

43. The method of claim 42, wherein the organism is a rodent or a primate.

44. The method of claim 42, wherein the organism is a human.

45. The method of claim 31, wherein the peptide library comprises fragments of a protein encoded by the genome of a first organism and the cells are derived from a second organism.

46. A method for identifying a peptide which modulates the infectivity of a pathogenic organism, said method comprising:
(a) providing a library of expression vectors, each of said vectors comprising a nucleic acid sequence which encodes a member of a peptide library, wherein the peptide library comprises fragments of one or more proteins which are encoded by the genome of the pathogenic organism;
(b) contacting a multiplicity of cells with the library of expression vectors under conditions suitable for transfection of the cells by the expression vectors and expression of the encoded peptide library within the cells;
(c) contacting the multiplicity of cells with the pathogenic organism;
(d) selecting a cell towards which the infectivity of the pathogenic organism is modulated; and
(e) determining the nucleic acid sequence of step (a) in the cell of step (c), wherein the peptide which is encoded by the nucleic acid sequence is identified as a peptide which modulates the infectivity of the pathogenic organism.

47. The method of claim 46, wherein the multiplicity of cells is derived from a mammal or an avian animal.

48. The method of claim 46, wherein the multiplicity of cells is derived from a primate or a rodent.

49. The method of claim 46, wherein the cells are derived from a human.

50. The method of claim 46, wherein the library of expression vectors comprises viral vectors.

51. The method of claim 46, wherein each vector further includes a regulatory sequence which is operatively linked to the nucleic acid sequence which encodes a member of a peptide library.

52. The method of claim 46, where the peptide library comprises fragments of two or more proteins encoded by the genome of the pathogenic organism.

53. The method of claim 52, wherein the peptide library comprises fragments of five or more proteins encoded by the genome of the pathogenic organism.

54. The method of claim 53, wherein the peptide library comprises fragments of ten or more proteins encoded by the genome of the pathogenic organism.

55. The method of claim 54, wherein the peptide library comprises fragments of fifteen or proteins encoded by the genome of the pathogenic organism.

56. The method of claim 46, wherein the pathogenic organism is a bacterium, a fungus, a protozoan or a virus.

57. A peptide which modulates a biological process identified according to the method of claim 1.

58. Use of a peptide which modulates a biological process identified according to the method of claim 1, for the molecular modeling of a compound having similar binding characteristics as said peptide.

59. A pharmaceutical composition comprising a peptide which modulates a biological process identified according to the method of claim 1, and a pharmaceutically acceptable carrier.

60. A method for treating a disease or condition associated with an aberrant biological process in a subject, comprising administering to the subject a therapeutically effective amount of a peptide which modulates a biological process identified according to the method of claim 1.

61. The method of claim 60, wherein the disease or condition is HIV infection.

62. The method of claim 60, wherein the disease or condition is cancer.

63. A kit for identifying a peptide which modulates a biological process comprising a peptide library comprising a multiplicity of peptides, wherein said peptides are fragments of at least one gene product of an organism and instructions for use.


SEQUENCE LISTING (6 sequences)

SEQ ID NO: 1 - DNA, 32 bp, Artificial Sequence
  oligonucleotide
tcgagccaccatgcacgtggtagctagctagc

SEQ ID NO: 2 - DNA, 32 bp, Artificial Sequence
  oligonucleotide
cggtggtacgtgcaccatcgatcgatcgccgg

SEQ ID NO: 3 - DNA, 33 bp, Artificial Sequence
  oligonucleotide
tcgagccaccatggcacgtggtagctagctagc

SEQ ID NO: 4 - DNA, 33 bp, Artificial Sequence
  oligonucleotide
cggtggtaccgtgcaccatcgatcgatcgccgg

SEQ ID NO: 5 - DNA, 34 bp, Artificial Sequence
  oligonucleotide
tcgagccaccatgggcacgtggtagctagctagc

SEQ ID NO: 6 - DNA, 34 bp, Artificial Sequence
  oligonucleotide
cggtggtacccgtgcaccatcgatcgatcgccgg